(19) United States
(12) Patent Application Publication
(10) Pub. No.: US 2006/0030794 A1
(43) Pub. Date: Feb. 9, 2006

(54) Human monitoring apparatus

(76) Inventors: Sarah Nation, Bloomington, IN (US); Jill Heinzen, Bloomington, IN (US); Joey Beyers, Bedford, IN (US); Tim Buckner, Bloomington, IN (US); Kurt Cavaggia, Bloomington, IN (US); Lowell Bailey, Bedford, IN (US); Rob Spencer, West Lafayette, IN (US); Kory Masterson, Bedford, IN (US); Timothy Moody, Bedford, IN (US); James David Ingalls, Bedford, IN (US); Jon Craton, Bedford, IN (US); Annie Bullock, Bedford, IN (US)

Correspondence Address: Intellectual Property Group;Bose McKinney & Evans LLP, 2700 First Indiana Plaza, 135 North Pennsylvania Street, Indianapolis, IN, 46204, US

(21) Appl. No.: 11/135,223

(22) Filed: May 23, 2005

Related U.S. Application Data
(60) Provisional application No. 60/573,678, filed on May 21, 2004.

(51) Int. Cl. A61B 5/103

(52) U.S. Cl. 600/593; 128/905; 600/595

(57) ABSTRACT

An apparatus used for monitoring a bodily function of a user. The apparatus includes a monitoring device, a communication line, a receiver, and a feedback device. The feedback device provides a feedback response to the user to remind them to repeat the bodily function.

### CROSS-REFERENCE TO RELATED APPLICATIONS

This application claims the benefit of U.S. Provisional Patent Application Ser. No. 60/573,678 to Heinzen et al., entitled Human Monitoring Apparatus (Attorney Docket No. 15911-0002), filed May 21, 2004.

### BACKGROUND

The present invention relates to monitoring human bodily functions. More particularly, the present invention relates to monitoring the frequency of swallowing and/or other bodily functions of a person.

### SUMMARY OF THE INVENTION

In accordance with one aspect of the present invention, a human monitoring apparatus is provided for monitoring a bodily function. The apparatus comprises a monitoring device, a communication line, a receiver, and a feedback device. The monitoring device is configured to detect when a bodily function occurs. The communication line is configured to communicate data based on a signal from the monitoring device. The receiver is configured to receive the data from the communication line. The feedback device is configured to indicate to a user to perform the bodily function.

According to another aspect of the present invention, a human monitoring apparatus for detecting a bodily function is provided. The human monitoring apparatus comprises a monitoring device, a communication line, a receiver, and a feedback device. The monitoring device is positioned adjacent a user's neck to detect when a bodily function occurs. The communication line is configured to communicate data based on a signal from the monitoring device. The receiver is operably coupled to the monitoring device by the communication line and configured to receive the data from the communication line. The receiver includes a timer to record an amount of time between each repetition of the bodily function. The feedback device indicates reminding the user to perform the bodily function.

### BRIEF DESCRIPTION OF THE DRAWINGS

A detailed description particularly refers to the accompanying figures in which:

FIG. 1 is a diagrammatic view of a person and a human monitoring apparatus showing the apparatus including a monitoring device positioned adjacent a neck of the person to monitor a bodily function of the person, a communication line coupled to the monitoring device, a receiver that receives communications from the communication line, and a feedback device also positioned adjacent the person's neck and coupled to the receiver by the communications line;

FIG. 2 is a diagrammatic view of a preferred circuit for the apparatus;

FIG. 3 a diagram showing operational steps of the apparatus; and

FIG. 4 is an alternative embodiment of the human monitoring apparatus similar to FIG. 1.

### DETAILED DESCRIPTION OF THE DRAWINGS

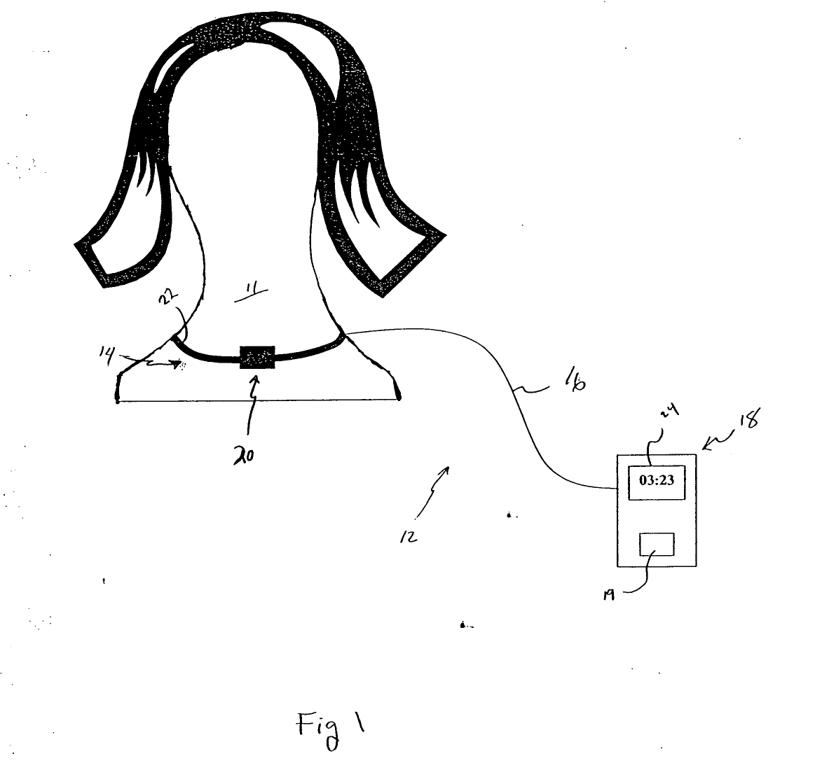

As shown in FIG. 1, a human monitoring apparatus 12 is provided for monitoring at least one bodily function of a user 11 such as swallowing, urinating, passing stool, breathing, or any other bodily functions known to those of ordinary skill in the art. On occasion, individuals with neurological or other disorders lack certain involuntary reflexes or impulses that control bodily functions such as swallowing. Other such individuals may fail to remember to urinate, pass stool, or to perform other similar bodily functions. Apparatus 12 monitors one or more of these bodily functions and reminds, stimulates, or teaches the individual to perform the bodily function.

According to the preferred embodiment of the present disclosure, apparatus 12 monitors if a person swallows within a predetermined time period. If the person does not swallow within the predetermined time period (typically 45-60 seconds), apparatus 12 reminds the person to swallow so that excess saliva does not collect in the person's mouth that might otherwise cause drool.

As shown in FIG. 1, apparatus 12 is provided for a person or user 11 to monitor a bodily function. Apparatus 12 includes a monitoring device 14 positioned adjacent the user's neck, a receiver 18, a communication line 16 extending between monitoring device 14 and receiver 18, and a feedback device 20 also positioned adjacent the user's neck. Alternatively, the feedback may be positioned at a location spaced from the neck similar to a pager. Monitoring device 14 detects or monitors one or more bodily functions of user 11. As shown in FIGS. 1-3, when a bodily function 21 occurs, device 14 detects this bodily function 21 and sends a signal through communication line 16 to receiver 18.

According to the preferred embodiment of the present disclosure, communication line 16 is a hardwired connection between device 14 and receiver 18. According to alternative embodiments of the present disclosure, communication is sent wirelessly to receiver 18 using known wireless transmission means or through any other transmission techniques known to those of ordinary skill in the art.

Receiver 18 includes a timer 17 that records the amount of time between each repetition of the bodily function. If timer 17 reaches a preset time between repetitions, a signal is sent to feedback device 20 which reminds the user to swallow or perform one or more other bodily functions. According to the preferred embodiment of the present disclosure, the preset time is adjustable by the user or another person.

After receiving the signal from monitoring device 14 that the user has performed the bodily function, receiver 18 resets timer 17. According to the preferred embodiment of the present disclosure, timer 17 may also be reset by an optional manual reset 19. For example, if apparatus 12 is being used to remind a person to urinate, the user may reset timer 17 each time the user urinates if monitoring device 14 fails to detect the urination.

If receiver 18 does not receive a signal from device 14 and timer 17 reaches the preset time, receiver 18 sends a feedback command to feedback device 20. Then feedback device 20 produces a feedback response to the user 11 to remind them to repeat the bodily function. According to the preferred embodiment that monitors swallowing, the feedback response produces a vibration on the user's neck to remind the user to swallow. According to other embodiments, the feedback response may be other audible, visual, electrical, or other physical stimuli to the individual notifying the individual that they must perform a bodily function.

According to the preferred embodiment of the present disclosure, shown in FIG. 1, monitoring device 14 includes a necklace 22 including a sensor (not shown). Preferably, the sensor is a strain gauge that measures expansion of the user's neck. However, any sensor operable to receive a signal will work. Other examples on sensors include a toggle switch, a Hall Effect sensor, and other field sensors that detect changes in an electric field due to changes in capacitance or inductance, other field sensors known to those of ordinary skill in the art, or any other sensor known to those of ordinary skill in the art. When the user swallows, their neck expands causing tension in necklace 22 which is exerted on the strain gauge. The tension on the strain gauge changes the electrical resistance of the strain gauge which is monitored by receiver 18 through communication line 16. Thus, the strain gauge provides an output signal to receiver 18 that can be monitored to indicate that the user has swallowed. Each time receiver 18 receives an indication from necklace 22 that the user has swallowed, receiver 18 resets timer 17.

According to the preferred embodiment of the present disclosure, receiver 18 includes an optional timer display 24 that provides a visual indication of how much time or timing increments have passed since the user's last repetition of the bodily function. For example, when monitoring swallowing, display 24 shows the amount of time since the user last swallowed. According to an alternative embodiment of the present disclosure, the display 24 provides a count down of the amount of preset time that is left before feedback device 20 is activated.

If receiver 18 does not receive an output signal from monitoring device 14 within the preset time, receiver 18 sends the feedback signal to feedback device 20 which reminds the user to perform the bodily function. According to the embodiment of present disclosure that monitors swallowing, feedback device 20 includes multiple vibrators supported by necklace 22 at different heights along the user's throat. The vibrators are sequentially activated to provide a stimulus to the user that progresses from the bottom of the throat to the top of the throat. According to alternative embodiments of the present disclosure, other feedback devices are provided such as a speaker controlled by a voice chip, an ear piece, a buzzer, an LED device, a vibrator positioned elsewhere on the user, or any other feedback device know to those skilled in the art and capable of providing at least one of an audible, visual or physical indications or stimulus.

According to one embodiment, feedback device 20 includes pair of eye glasses, an LED, and fiber optic line transmitting light from the LED to the pair of glasses. The fiber optic line is positioned around the lenses of the glasses on the rims. Notches are provided in the fiber optic line so that light “bleeds” out of the line and can be seen by the user wearing the glasses. Receiver 18 activates the LED when timer 17 reaches the preset time. The light generated by the LED is piped through the fiber optic line. The user then sees the light bleeding through the notches and knows that it is time to repeat the bodily function, such as swallowing.

According to an alternative embodiments of the present disclosure, monitoring device 14 includes a mouthpiece that measures the temperature in the mouth of the user. The mouth piece provides a wireless signal to receiver 18 if the saliva temperature reaches a threshold temperature. This threshold temperature can be an indication that saliva has collected in the user's mouth and it is time to swallow to avoid collecting too much saliva. Based on the signal from the mouth piece, receiver 18 activates feedback device 20 to remind the user to swallow.

According to another embodiment of the present disclosure, monitoring device 14 is a microphone (not shown) positioned at a location to monitor noises made by the user's body that are indicative of when a bodily function has or has not occurred. When monitoring swallowing, the microphone is positioned at a location to monitor noise made by a user's throat when the user swallows. The microphone sends signals to receiver 18. Receiver 18 recognizes the frequency the user's throat makes when swallowing. Each time the user swallows and receiver 18 recognizes the swallowing frequency, timer 17 is reset.

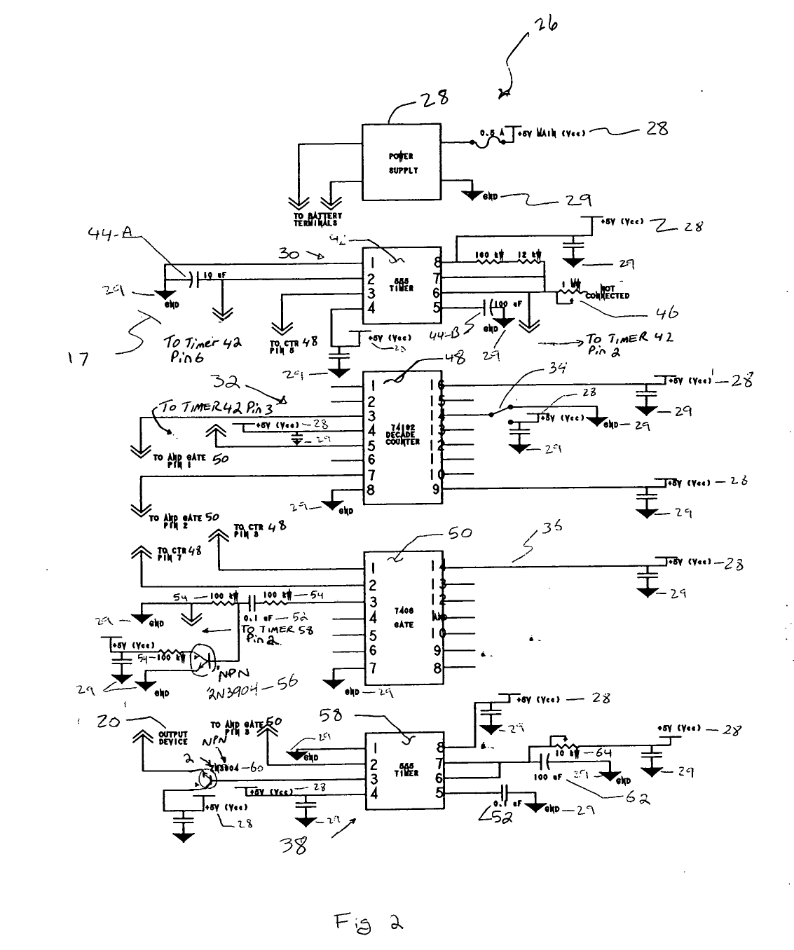

As shown in FIG. 2, receiver 18 includes a control circuit or controller 26 that includes a power source 28, ground 29, timer 17 including a timing device 30 and a recorder or counter 32, a switch 34 electrically coupled to the preferred strain gauge (not shown) and manual reset 19, a gate 36 electrically coupled to display 24, and a second timer 38 electrically coupled to feedback device 20. Controller 26 may also be embedded anywhere on monitoring apparatus 12, not just in receiver 18.

According to one embodiment, power source 28 is a 5 volt battery. First timer 30 includes a “555 timer” 42, a 10 μF capacitor 44-A and a 100 μF capacitor 44-B, a 160 KΩ resistor, a 12 KΩ resistor, and a 1MΩ potentiometer 46. Counter 32 includes a “74192 Decade Counter” 48 set to a predetermined value. Gate 36 includes a “7408 AND Gate” 50, a 0.1 μF capacitor 52, three 100 KΩ resistors 54, and a first 2N3904 UJT transistor 56. Second timer 38 includes a second “555 Timer” 58, a 0.1 μF capacitor 52, a second 2N3904 UJT transistor 60, a 100 μF capacitor 62, and a 10 KΩ potentiometer 64.

In operation, an operator (not shown) sets the predetermined time period by adjusting potentiometer 46. An adjustment to potentiometer 46 adjusts the timer period for first timer 30. When first timer 30 reaches its predetermined time period it sends a signal to counter 32. Signals from first timer 30 and counter 32 are sent to gate 50 and amplified by first transistor 56 to appear on display 24. If counter 32 reaches its predetermined counter value prior to being reset by the strain gauge (indicating swallowing) or tripping of manual reset 19, counter 32 sends a signal to second timer 38. After the signal is boosted by second transistor 60, second timer 38 provides a signal to feedback device 20 to remind the user to perform the bodily function. Second timer 38 provides the signal for a predetermined duration which may be adjusted by the operator by adjusting potentiometer 64.

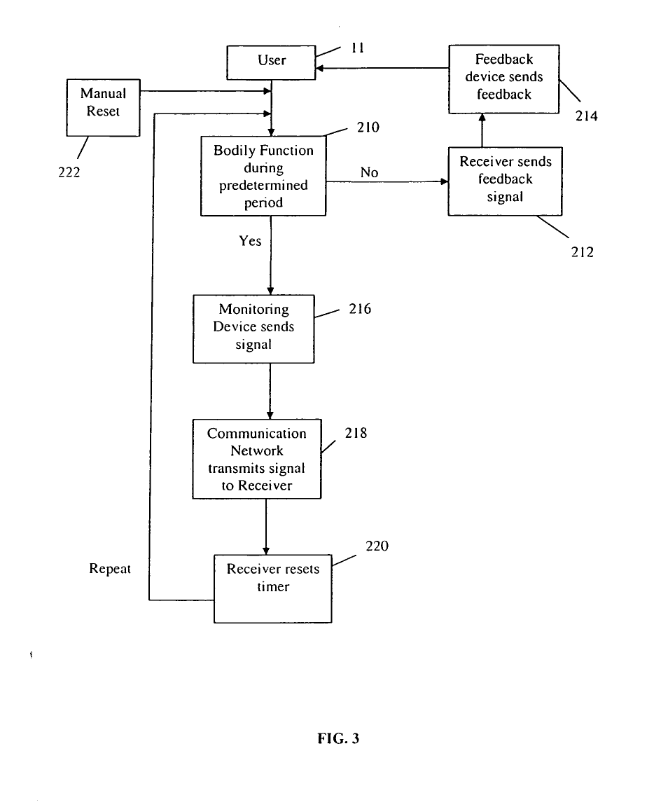

Operational steps of the apparatus are shown in FIG. 3. After the user 11 places the monitoring apparatus 12 on their person, the monitoring apparatus 12 determines 210 if a bodily function occurred during a predetermined time. If no, the receiver sends 212 a feedback signal to the feedback device. The feedback device sends 214 feedback to the user 11. If yes, the monitoring device sends 216 a signal to the communication network. The communication network transmits 218 the signal to the receiver. The receiver then resets 220 the timer and repeats the system. Optionally, a manual reset may be activated 222 by the user 11 or an operator.

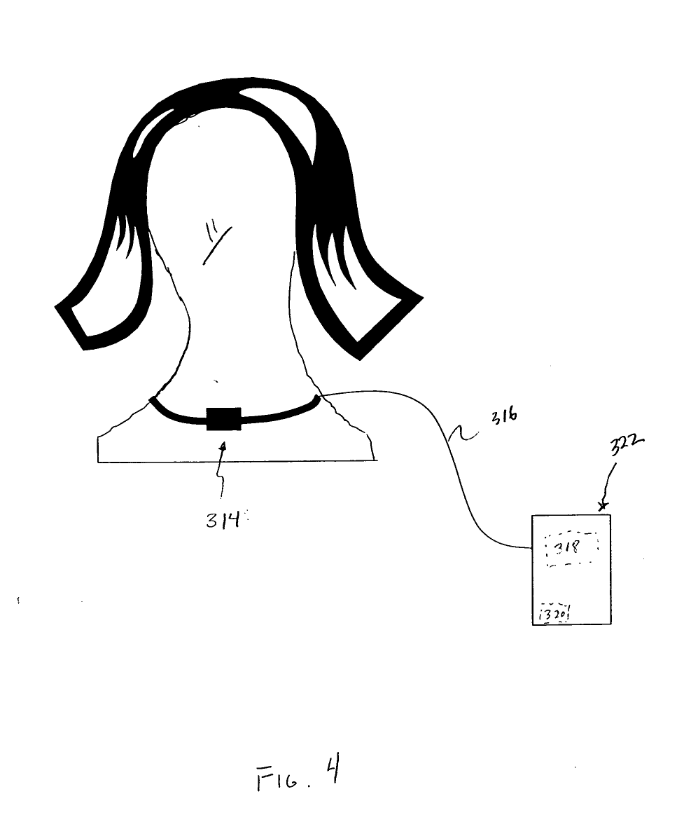

An alternative embodiment of the present invention is shown in FIG. 4.

A monitoring apparatus 312 is provided for a person or user 11 to monitor a bodily function. Apparatus 312 includes a monitoring device 314 positioned adjacent the user's neck, a receiver 318, a communication line 316 extending between monitoring device 314 and receiver 318, and a feedback device 320. Receiver 318 and feedback device are provide within a housing 322 positioned at a location spaced from the neck similar to a pager. Monitoring device 314 detects or monitors one or more bodily functions of user 11. When a bodily function 321 occurs, device 314 detects this bodily function 321 and sends a signal through communication line 316 to receiver 318. The components operate in a similar fashion to those discussed above in the preferred embodiment and may be interchangeable with previously discussed embodiments.

Although the present invention has been described with reference to particular means, materials and embodiments, from the foregoing description, one skilled in the art can easily ascertain the essential characteristics of the present invention and various changes and modifications may be made to adapt the various uses and characteristics without departing from the spirit and scope of the present invention. It should be understood, however, that there is no intent to limit the invention to the particular forms disclosed, but on the contrary, the intention is to cover all modifications, equivalents, and alternatives falling within the spirit and scope of the invention as defined by the appended claim.

1. A human monitoring apparatus comprising,
a monitoring device configured to detect when a bodily function occurs;
a communication line configured to communicate data based on a signal from the monitoring device;
a receiver configured to receive the data from the communication line; and
a feedback device configured to indicate to a user to perform the bodily function.

2. The human monitoring apparatus of claim 1, wherein the bodily function is swallowing.

3. The human monitoring apparatus of claim 1, wherein the receiver includes a power source, a timer, and a switch.

4. The human monitoring apparatus of claim 3, wherein the power source is a battery.

5. The human monitoring apparatus of claim 3, wherein the switch is operably coupled to the monitoring device.

6. The human monitoring apparatus of claim 3, wherein each time the receiver receives the signal from the monitoring device the timer resets.

7. The human monitoring apparatus of claim 1, wherein the monitoring device includes a necklace having a sensor.

8. The human monitoring apparatus of claim 7, wherein the sensor is a strain gauge to measure expansion of a user's neck.

9. The human monitoring apparatus of claim 8, wherein expansion of the user's neck causes tension in the necklace which is exerted on the strain gauge.

10. The human monitoring apparatus of claim 7, wherein the monitoring device includes a switch that is triggered by the expansion of the user's neck.

11. A human monitoring apparatus comprising,
a monitoring device positioned adjacent a user's neck to detect when a bodily function occurs;
a communication line configured to communicate data based on a signal from the monitoring device;
a receiver operably coupled to the monitoring device by the communication line and configured to receive the data from the communication line, the receiver including a timer that records an amount of time between each repetition of the bodily function; and
a feedback device to indicate to the user to perform the bodily function.

12. The human monitoring apparatus of claim 11, wherein the feedback device is positioned adjacent the user's neck.

13. The human monitoring apparatus of claim 11, wherein the feedback device is positioned spaced from the user's neck.

14. The human monitoring apparatus of claim 11, wherein when the bodily function occurs the monitoring device detects the bodily function and sends a signal through the communication line to the receiver.

15. The human monitoring apparatus of claim 11, wherein the communication line is a hardwired connection between the monitoring device and the receiver.

16. The human monitoring apparatus of claim 11, wherein the communication line is a wireless connection between the monitoring device and the receiver.

17. The human monitoring apparatus of claim 11, wherein if the timer reaches a preset time between repetitions, the feedback device indicates to the user to perform the bodily function.

18. The human monitoring apparatus of claim 17, wherein the preset time is adjustable by at least one of the user and a different person.
